(19) United States
(12) Patent Application Publication
(10) Pub. No.: US 2011/0106086 A1
(43) Pub. Date: May 5, 2011

(54) TOOL JIG FOR BONE IMPLANT ASSEMBLY

(75) Inventor: Ewen Laird, New South Wales (AU)

(73) Assignee: AUSTOFIX GROUP LIMITED, North Plympton (AU)

(21) Appl. No.: 12/936,195

(22) PCT Filed: Apr. 3, 2009
(86) PCT No.: PCT/AU09/00410

§ 371 Date: Dec. 23, 2010

(30) Foreign Application Priority Data
Apr. 3, 2008 (AU) 2008901592

(51) Int. Cl. A61B 17/80; A61B 17/56

(52) U.S. Cl. 606/70; 606/96

(57) ABSTRACT

The invention provides a tool jig (50) for use with a bone plate (10) having one or more fixing element apertures (20), wherein the tool jig (50) guides placement of a drill sleeve (34) to enable the drill sleeve (34) to be attached to a fixing element aperture (20), the tool jig (50) comprising a body (52), a securing means (80) on the body adapted to releasably secure the tool jig (50) to the bone plate (10) such that the tool jig (50) can be firstly secured in position to the bone plate (10) and secondly removed, and one or more tool jig apertures (54) defined in the body (52) wherein an at least one or all tool jig apertures (54) are adapted to be substantially aligned with an at least one or all fixing element apertures (20); wherein, in use, the tool jig aperture (54) guides the drill sleeve (34) so that the longitudinal axis of the drill sleeve (34) substantially aligns with the axis of the fixing element aperture (20) prior to attaching the drill sleeve (34) to the fixing element aperture (20). The invention also provides a medical implant assembly (5) comprising the tool jig (50) and the bone plant (10), a kit comprising the medical implant assembly and at least one drill sleeve (34), and methods of using same.

### FIELD OF THE INVENTION

The present invention relates to a medical implant assembly. In particular, the invention relates to a medical implant assembly that is fixed to bone. In a particular embodiment, the invention relates to a tool jig for a bone implant assembly.

### PRIORITY DOCUMENTS

The present application claims priority from Australian Provisional Patent Application No. 2008901592 titled “Medical Implant Assembly” and filed on 3 Apr. 2008.

### BACKGROUND OF THE INVENTION

It is advantageous that a fractured bone is stabilised as quickly as possible to achieve adequate healing of a fracture. To this end, implantable bone plates can be utilised to hold a fractured bone in an optimised position to enhance healing. The bone plate can be firmly fixed to the bone in the vicinity of the fracture site by means of screws and other fixing elements that pass through a bone plate aperture, for example, utilising a series of preformed holes drilled into the bone prior to bone plate insertion. It is desirable that each hole drilled into the bone is positioned and orientated precisely in accordance with each bone plate aperture in order to maximise the chance the fracture healing well.

However, it is difficult to pre-drill holes in bone in precisely the right position and orientation. Further, surgery and anaesthesia can be problematic or life-threatening for many patients, and it is accordingly desirable that the said procedure is optimised to be as simple and short in duration as practical in order to minimise risk to the patient.

A drill jig can guide a drill, or drill sleeve guiding a drill, or similar, to increase the accuracy of positioning of holes drilled into bone at a fracture site. However, surgeons may need to hold a drill jig in place manually, resulting in inaccuracies in alignment of the drill jig and the bone plate. Alternatively, the drill jig may be fixed to the bone plate prior to implantation of the bone plate, for example, using screws or the like. However, it is still difficult to drill a hole into bone on the desired angle using these drill jigs fixed to the bone plate, as such drill jigs permit some inaccuracies in alignment of the drill or drill sleeve, for example, permitting flexibility in the angle of the drill or drill sleeve, or possibly resulting in cross threading of the drill hole when the fixing element is inserted.

It is an object of the present invention to overcome at least some of these problems by providing an improved tool jig that aligns a drill sleeve within a fixing element aperture in a bone plate such that a hole can be drilled into bone with improved accuracy.

### SUMMARY OF THE INVENTION

The present applicant has realised that drilling sleeves can be firmly held by an aligned tool jig aperture within a bone plate aperture in a manner that permits drilling of a hole in bone in a predetermined manner.

Accordingly, in a first aspect, the present invention provides a tool jig for use with a bone plate having one or more fixing element apertures, wherein the tool jig guides placement of a drill sleeve to enable the drill sleeve to be attached to a fixing element aperture, the tool jig comprising
a body,
a securing means on the body adapted to releasably secure the tool jig to the bone plate such that the tool jig can be firstly secured in position to the bone plate and secondly removed, and
one or more tool jig apertures defined in the body wherein an at least one or all tool jig apertures are adapted to be substantially aligned with an at least one or all fixing element apertures;
   wherein, in use, the tool jig aperture guides the drill sleeve so that the longitudinal axis of the drill sleeve substantially aligns with the axis of the fixing element aperture prior to attaching the drill sleeve to the fixing element aperture.

The term “bone plate” is intended to mean any suitable plate that can be fixed to a bone about a bone fracture site that strengthens, stabilises and/or aids fixation of the bone. The bone plate may be used on bone from a variety of regions on the body (eg hands, feet, arms and legs). In an embodiment, the bone plate may be specifically adapted to fix a particular bone or bone joint, for example, a hip joint bone (eg the head of the femur, the greater trochanter), a wrist bone (eg the distal radius bone), a femur bone, a humerus bone, a tibia bone, an ulna bone, etc. For example, the bone plate may be adapted to conform to the shape of the anatomy of a particular bone or joint, and/or it may have fixing element apertures that are designed to enable fixing elements to be inserted into the bone at the most appropriate angles to enhance fixing of fractures of that particular bone or joint.

The term “bone” is not limited to a single bone and may include a series of bones.

The tool jig of the present invention advantageously provides a means for aligning a drill sleeve within a fixing element aperture in a bone plate such that a hole can be drilled into bone in a predetermined position and/or angle. Further, the alignment of the drill sleeve by the tool jig aperture within the fixing element aperture advantageously decreases the risk of cross-threading between a fixing element and a drilled hole in bone. Thus, the use of the tool jig of the present invention advantageously simplifies and hastens surgery to implant a bone plate. Additionally, the use of the tool jig of the present invention offers a means to improve the positioning of a bone plate such that fixing elements are inserted into bone in a predetermined manner that can advantageously maximise the chance of a fracture healing well.

Preferably, the drill sleeve is a cylindrical drill sleeve for guiding a drill to drill a hole in bone.

Preferably, the securing means extends from the body and is adapted to, in use, resiliently engage with the bone plate by clipping to releasably secure the tool jig to the bone plate. The securing means can advantageously provide a convenient and simple means to clip (eg by resilient snap fastening) the tool jig to the bone plate in a manner that substantially reduces movement of the tool jig relative to the bone plate, without the need to remove fixing elements such as screws during surgery, enabling the surgery to be quicker and simpler. It is to be understood that once the tool jig is releasably secured to the bone plate by clipping, there is substantially no movement of the tool jig relative to the bone plate. Advantageously, once the tool jig is releasably secured to the bone plate by clipping, the chance of the tool jig being accidentally dislodged from the bone plate during surgery is minimised, as removal of the tool jig from the bone plate requires the application of some force.

The securing means may extend from the body to a securing region having at least one securing tooth member that projects from the securing region to, in use, engage with an engaging surface of the bone plate to releasably secure the tool jig to the bone plate. The securing means may comprise a plurality of finger members. Preferably, at least two opposed securing tooth members each comprise a flange means that, in use, engage with an indented securing surface of the bone plate to releasably secure the tool jig to the bone plate.

The term “indented securing surface” is intended to refer to a region on the bone plate which is inwardly angled or recessed such that, in use, a securing tooth member projects to and conforms with an indented securing region to secure the tool jig to the bone plate. The term “flange means” is intended to refer to a protrusion that projects from the securing tooth members such that, in use, the flange means extends into the indented securing surface to secure the tool jig to the bone plate.

Preferably, the tool jig further comprises a recessed region that, in use, is adapted to allow engagement with a tool that enables separation of the tool jig from the bone plate. In an embodiment, the recessed region is located at the base of the tool jig. Alternatively, the recessed region is at least one of the tool jig apertures.

In an embodiment, at least one of the tool jig apertures further guides placement of an outer sleeve within the tool jig aperture, wherein the outer sleeve further guides placement of the drill sleeve within the fixing element aperture. Preferably, the outer sleeve is also a tool that enables separation of the tool jig from the bone plate.

In an embodiment, at least one or all of the tool jig apertures have an internal diameter that is adapted to have a sliding fit with the drill sleeve or an outer sleeve for guiding a drill sleeve. In another embodiment, at least one or all of the tool jig apertures are at least partially threaded to engage the drill sleeve or an outer sleeve for guiding a drill sleeve. In yet another embodiment, at least one or all of the tool jig apertures have an internal diameter that is adapted to be larger than the external diameter of a fixing element such that, in use, the fixing element can pass through the tool jig aperture to be seated within the body plate aperture.

Preferably, the tool jig apertures are adapted such that, in use, each tool jig aperture is aligned with a predetermined fixing element aperture of a predetermined bone plate. More preferably, the tool jig is adapted to be used with a bone plate selected from a fixed angle distal radius bone plate or a hip bone plate.

In a second aspect, the present invention provides a medical implant assembly comprising:
a tool jig according to the first aspect of the invention, and
a bone plate having one or more fixing element apertures adapted to receive a drill sleeve;
   wherein the tool jig aperture acts to guide the drill sleeve so that the longitudinal axis of the drill sleeve substantially aligns with the axis of the fixing element aperture prior to attaching the drill sleeve to the fixing element aperture.

Preferably, the bone plate is selected from a fixed angle distal radius bone plate or a hip bone plate.

In a third aspect, the present invention provides a kit comprising the medical implant assembly according to the second aspect of the invention and at least one drill sleeve. In an embodiment, the kit comprises the medical implant assembly, an at least one drill sleeve and an at least one outer sleeve. Preferably, the kit contains all of the drill sleeves and/or outer sleeves that are required to fix the predetermined bone plate to the bone.

In a fourth aspect, the present invention provides a method of fixing a bone plate to a bone comprising the following steps:
(a) arranging the medical implant assembly of the second aspect of the invention against a bone;
(b) inserting a drill sleeve into a tool jig aperture and attaching the drill sleeve to the fixing element aperture wherein the tool jig aperture guides the drill sleeve so that the longitudinal axis of the drill guide substantially aligns with the axis of an aligned fixing element aperture;
(c) drilling a hole in a predetermined position in the bone by locating the drill within the drill sleeve;
(d) removing the tool jig from the bone plate; and
(e) inserting a fixing element into the fixing element aperture and the hole that has been drilled to fix the bone plate to the bone.

In a fifth aspect, the present invention provides a method for fixing a bone plate to a bone comprising the following steps:
(a) arranging the medical implant assembly of the second aspect of the invention against a bone;
(b) inserting a drill sleeve into a tool jig aperture and attaching the drill sleeve to the fixing element aperture wherein the tool jig aperture guides the drill sleeve so that the longitudinal axis of the drill guide substantially aligns with the axis of an aligned fixing element aperture;
(c) drilling a hole in a predetermined position in the bone by locating the drill within the inner drill sleeve;
(d) removing the drill sleeve;
(e) inserting a fixing element into the fixing element aperture and the hole that has been drilled to fix the bone plate to the bone; and
(f) removing the tool jig from the bone plate.

In a sixth aspect, the present invention provides a method for fixing a bone plate to a bone comprising the following steps:
(a) arranging the medical implant assembly of the second aspect of the present invention against a bone;
(b) threading an outer sleeve into a tool jig aperture wherein the tool jig aperture guides the outer sleeve so that the longitudinal axis of the outer sleeve substantially aligns with the axis of an aligned fixing element aperture;
(c) slidably locating an inner drill sleeve within the outer sleeve and attaching the drill sleeve to the aligned fixing element aperture;
(d) drilling a hole in a predetermined position in the bone by locating the drill within the inner drill sleeve;
(e) removing the inner drill sleeve;
(f) inserting a fixing element through the outer sleeve and into the aligned fixing element aperture and the hole that has been drilled to fix the bone plate to the bone; and
(g) removing the tool jig from the bone plate using the outer sleeve as a tool.

A specific embodiment of the invention will now be described in some further detail with reference to and as illustrated in the accompanying figures. This embodiment is illustrative, and is not meant to be restrictive of the scope of the invention.

### BRIEF DESCRIPTION OF THE DRAWINGS

An illustrative embodiment of the present invention will be discussed with reference to the accompanying drawings wherein:

FIG. 1 shows an embodiment of a top perspective view of a medical implant assembly;

FIG. 2 shows an embodiment of a tool jig, with FIG. 2A showing a bottom view, FIG. 2B shows a rearward end view, FIG. 2C shows a plan view, FIG. 2D shows a top perspective view, and FIG. 2E shows a bottom perspective view;

FIG. 3 shows an embodiment of a tool jig prior to engagement with a bone plate;

FIG. 4 shows the embodiment of FIG. 3 of a tool jig releasably secured with a bone plate to form a medical implant assembly;

FIG. 5 shows the embodiment of FIGS. 3 and 4 of a tool jig after disengagement with a bone plate;

FIG. 6 shows the embodiment of FIGS. 3 to 5 illustrating a step in a method for fixing a bone plate with bone wherein a drill guide is inserted into the tool jig aperture to be aligned with and attached to the fixing element aperture;

FIG. 7 shows the embodiment of FIGS. 3 to 5 illustrating a further step in a method for fixing a bone plate with bone wherein a hole is drilled in a predetermined position in bone by locating the drill within the drill sleeve;

FIG. 8 shows the embodiment of FIGS. 3 to 5 illustrating a further step in a method for fixing a bone plate with bone wherein a fixing element is inserted through the tool jig aperture into the fixing element aperture;

FIG. 9 shows the bone plate fixed to bone following removal of the tool jig in the embodiment shown in FIGS. 3 to 8;

FIG. 10 shows an embodiment of a medical implant assembly in a side perspective view;

FIG. 11 shows the embodiment of FIG. 10 of a medical implant assembly in a top perspective view;

FIG. 12 shows the embodiment of FIGS. 10 and 11 of a tool jig prior to engagement with a bone plate in a side perspective view;

FIG. 13 shows the embodiment of FIGS. 10 to 12 a tool jig releasably secured with a bone plate to form a medical implant assembly in a side perspective view;

FIG. 14 shows the embodiment of FIGS. 10 to 13 illustrating an exploded view of the bone plate, tool jig, inner drill sleeve and outer sleeve in a side perspective view;

FIG. 15 shows the embodiment of FIGS. 10 to 14 illustrating a step in a method for fixing a bone plate with bone wherein a drill is inserted through the inner drill sleeve which is located within an outer sleeve which is located within the tool jig aperture in a side perspective view;

FIG. 16 shows the embodiment of FIGS. 10 to 15 illustrating a further step in a method for fixing a bone plate with bone wherein a fixing element is about to be inserted through the outer sleeve which is located within the tool jig aperture in a side perspective view; and

FIG. 17 shows the bone plate of the embodiment of FIGS. 10 to 16 fixed to bone following removal of the tool jig.

In the following description, like reference characters designate like or corresponding parts throughout the several views of the drawings.

### DESCRIPTION OF ILLUSTRATIVE EMBODIMENTS

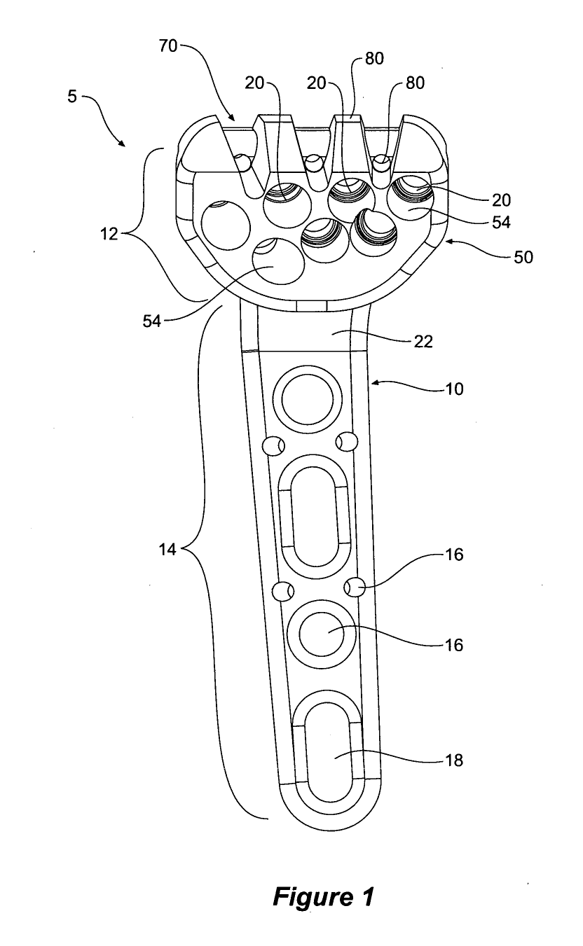

Referring to FIG. 1, there is shown a medical implant assembly 5 comprising a tool jig 50 which is releasably secured to a bone plate 10. The bone plate 10 consists of a head portion 12 and tail portion 14 encompassing a plurality of fixing element apertures 16,18,20. In use, fixing elements are inserted through the fixing element apertures into underlying bone 30 to fix the bone plate 10 to bone 30. The fixing element apertures 16,18,20 are located in predetermined positions and set at predetermined angles to enable fixing elements 32, 36 to be placed into the bone 30 in positions and angles that, together with the bone plate 10, optimally enhance healing of a fractured or broken bone. The fixing elements 32,36 may engage bone fragments, fractured bone or non-fractured bone. The fixing element apertures 16,18,20 within the bone plate 10 are elongate fixing element apertures 18 or circular fixing element apertures 16,20 and are sized to fit a range of fixing elements such as but not limited to screws, nails, pins and Kirschner wires. The fixing element apertures 16,18,20 may be threaded, partially threaded or plain; and they may be shaped or tapered such that fixing elements can be seated flush within the aperture.

When the tool jig 50 is releasably secured to the bone plate 10, the tool jig apertures 54 precisely align with the predetermined positions and angles of the fixing element apertures 20. Preferably, fixing element apertures 20 are sufficiently sized to receive orthopaedic screws. Fixing element apertures 20 are also adapted such that a drill sleeve 34 can be attached, for example, by being threaded or having a bayonet connection. In the illustrated embodiment, the fixing element apertures 20 are threaded so that, in use, a drill sleeve 34 can be attached into the fixing element apertures 20 by threading the drill sleeve 34 into the threaded fixing element aperture 20. The drill sleeve 34 and the bone plate 10 are arranged such that the drill sleeve 34 extends a predetermined distance from the bone plate 10 though the tool jig aperture 54 once drill sleeve 34 has been completely screwed in and tightened. This substantially results in a predetermined drilling depth in which fixing elements of a predetermined length can be used to fix the bone plate 10 to underlying bone 30.

The internal diameter of the tool jig apertures 54 is adapted such that, in use, a drill sleeve 34 have a sliding fit within a tool jig aperture 54. In use, a drill sleeve 34 is inserted in the tool jig aperture 54, and the tool jig aperture 54 guides the drill sleeve 34 into the fixing element aperture 20 so that the longitudinal axis of the drill sleeve 34 substantially aligns with the axis of the fixing element aperture 20 in the bone plate 10 prior to attaching the drill sleeve 34 to the fixing element aperture 20. By this means, the tool jig apertures 54 align drill sleeves 34 at the correct angle and direct the drilling of holes into the underlying bone 30 in a predetermined position and angle within the fixing element apertures 20 such that fixing elements can be placed at predetermined positions in the bone 30. The insertion of fixing elements at various predetermined angles in some bones may provide an enhanced retention force of the bone plate to bone and/or engage small bone fragments.

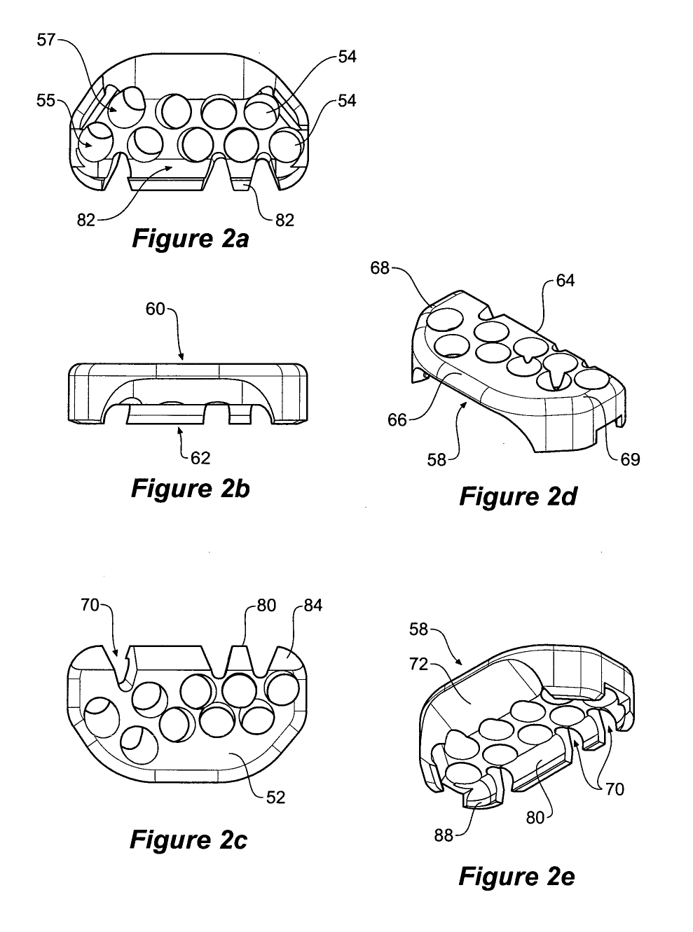

Referring to FIGS. 2A to 2E, there is shown a tool jig 50 comprising a body 52 encompassing a plurality of tool jig apertures 54, securing means comprising finger members 80, and a recess 58. The tool jig apertures 54 are set at predetermined positions and angles such that in use each tool jig aperture precisely aligns with a fixing element apertures 20. Accordingly, the illustrated embodiment shows tool jig apertures 54 located in two rows 55,57, and the tool jig apertures 54 are angled or normal to the bone plate depending upon the angle of the corresponding fixing element aperture 20. The tool jig apertures 54 pass from a top 60 through to a base 62, although some or all of the tool jig apertures 54 may pass through different surfaces of the tool jig 50 in some embodiments. The tool jig apertures 54 in the illustrated embodiment are non-threaded and substantially cylindrical in shape, with an equal diameter at the top 60 and base 62 of the tool jig 50. However, in some embodiments, the tool jig apertures 54 may be threaded or be partially threaded, or be shaped or tapered. In some embodiments, the diameter of the apertures 54 is such that fixing elements can pass through the tool jig 50 to be seated within the fixing element apertures 20.

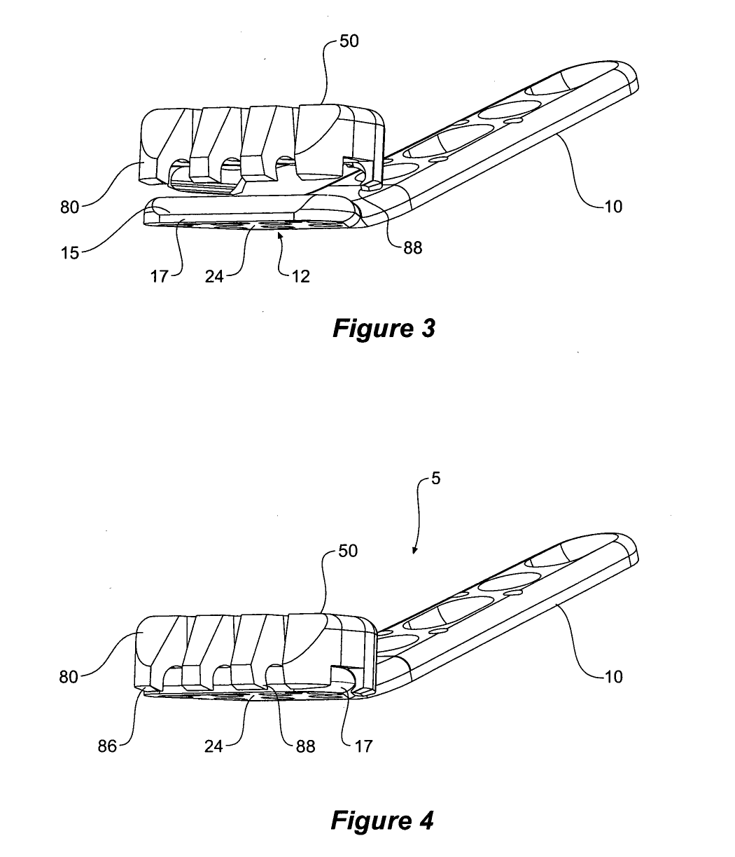

The tool jig 50 has a securing means which is adapted to releasably secure the tool jig 50 to the bone plate 10 by clipping, and after the drilling of holes through drill guides 34 aligned by the tool jig apertures 54 into fixing element apertures 20, the tool jig 50 can be released from the bone plate 10. The securing means includes a plurality of finger members 80 that extend from the body 52 of the tool jig 50 and resiliently engage with the bone plate 10. The engaging surface 15 on the perimeter of the head 12 of bone plate 10, and finger members 80, are contoured to engage with one another to releasably secure the tool jig 50 to the bone plate 10 in a manner that substantially prevents movement of the tool jig 50 relative to the bone plate 10. As illustrated in FIGS. 2 to 4, the interior aspects 82 of the finger members 80 are concavely shaped to conform and engage with the engaging surface 15 of the bone plate 10 such that in use the finger members 80 secure the tool jig 50 to the bone plate 10 by clipping. The finger members 80 are positioned on the perimeter of the body 52 of the tool jig 50. In particular, the finger members 80 are located on the forward end 64 and at the corners of the body 52 between the rearward end 66 and sides 68,69 of the tool jig 50. The finger members 80 extend from the top 60 to the base 62 of the tool jig 50 and are integrally formed with the body of the tool jig 50. A securing region is located at the base 86 of each finger member 80, and a securing tooth member 88 projects from the securing region to, in use, engage with the engaging surface 15 of the bone plate 10. As shown in FIGS. 3 and 4, the engaging surface 15 includes an indented securing surface 17, which is an inwardly angled surface at the perimeter of base 24, and which is contoured for engagement with securing tooth member 88. In the illustrated embodiment, the base 86 of the finger members 80 abuts with the base 24 of the head 12 of the bone plate 10. Securing tooth members 88 may comprise a flange means that in use engages with the indented securing surface of the bone plate 10. In use, the finger members 80 resiliently deform to deflect outwardly to allow engagement of the tooth member 88 with the indented securing surface 17 to thereby securely clip the tool jig 50 onto the bone plate 10.

In some embodiments, gaps 70 are located between finger members 80. Access to some fixing element apertures 16 in the bone plate 10 may be enabled through gaps 70. The fixing element apertures 16 may be accessed by a component including a drill sleeve, an outer sleeve, a drill and/or a fixing element, to enable the positioning of fixing elements within element apertures 16. For example, it may be possible to fix the medical implant assembly 5 to the bone 30 by drilling a hole through the bone underlying fixing element aperture 16 in the gaps 70, and then insert a fixing element into fixing element apertures 16 to fix the assembly to bone prior to drilling of holes through drill guides 34 aligned by the tool jig apertures 54 into fixing element apertures 20.

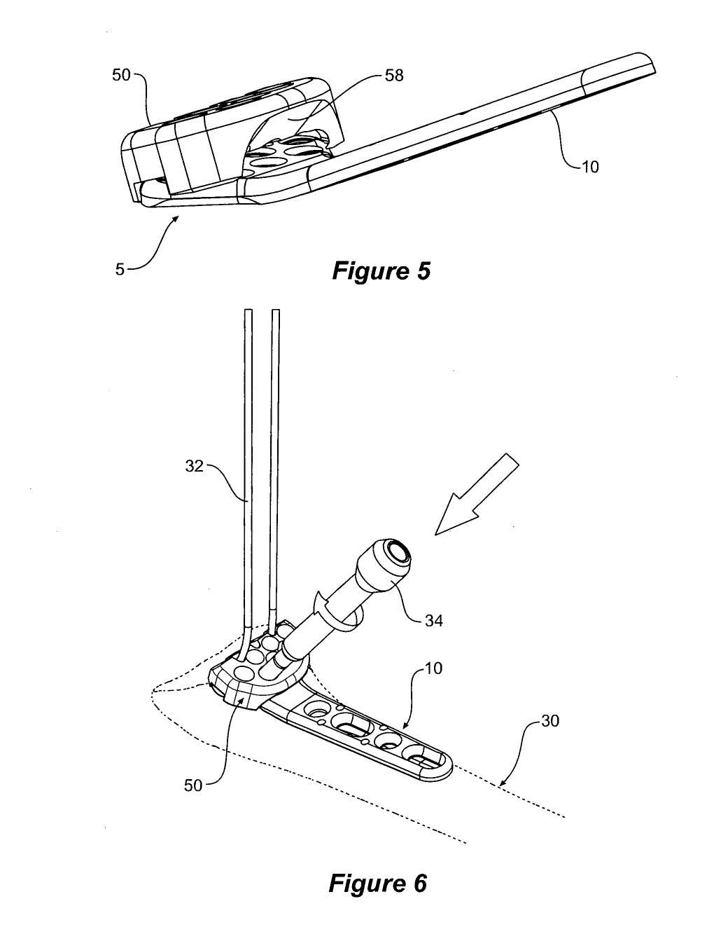

When the tool jig 50 is releasably secured with bone plate 10, the application of some force is required to remove the tool jig 50 from bone plate 10, minimising the possibility of accidental dislodgment during surgery. Accordingly, the tool jig 50 may further comprise a recessed region that, in use, is adapted to allow engagement with a tool that enables separation of the tool jig 50 from the bone plate 10. In the illustrated embodiment, a recessed region 58 is located at the base 62 of the tool jig 50. It extends along the rearward end 66 of the tool jig 50 to the finger members 80 located at corners of the body 52 between the rearward end 66 and sides 68,69 of the tool jig 50. The depth of the recessed region is such that the angled surface 72 is terminated in the vicinity of the second row of apertures 55, closest to the forward end 64 of the tool jig 50. As such, the recessed region 58 is sufficient to enable a tool such as a lever (not shown) to be inserted into the recess 58 to apply a sufficient separating force to separate the tool jig 50 from the bone plate 10 as shown in FIG. 5.

In another embodiment (not illustrated), a recessed region is located on the top face of the tool jig. The recessed region is sufficiently sized to enable a tool element to be inserted into the recess to lever or lift the tool jig and disengage the tool jig from the bone plate. The tool may be any suitable tool including a lever or a drill guide.

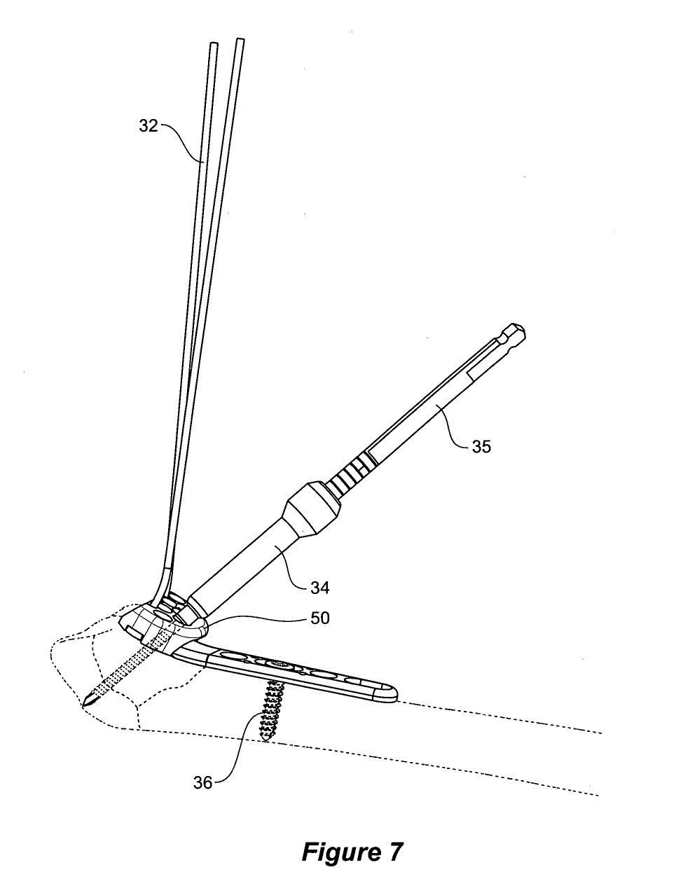

FIGS. 4 to 8 illustrate a method of method of fixing the bone plate 10 to a bone 30. The medical implant 5 may be assembled by clipping the tool jig 50 onto the bone plate 10 such that the tooth members 88 of the finger members 80 engage the engaging surface 15 of the bone plate 10. The medical implant assembly may be sterilised prior to implant surgery. During surgery, the medical implant assembly 5 is arranged against the bone 30. The bone plate 10 may be provisionally engaged with the underlying bone by inserting fixing elements 32, 26 though holes drilled through fixing element apertures 16,18. In some embodiments, Kirschner wires 32 are used to engage bone fragments (FIG. 6). A drill sleeve 34 is inserted into a tool jig aperture 54 and the drill sleeve 34 is attached to the fixing element aperture 20, such that the tool jig aperture 54 guides the drill sleeve 34 so that the longitudinal axis of the drill guide 34 substantially aligns with the axis of an aligned fixing element aperture 20. A drill 35 is inserted within the drill sleeve 34 (FIG. 7) and a hole is accordingly drilled in a predetermined position in the bone 30. The tool jig 50 may then be removed from the bone plate 10 by inserting a lever into the recessed region 58 and applying a separating force. A fixing element 36 is then inserted into the fixing element aperture 20 and the hole that has been drilled to fix the bone plate to the bone 30.

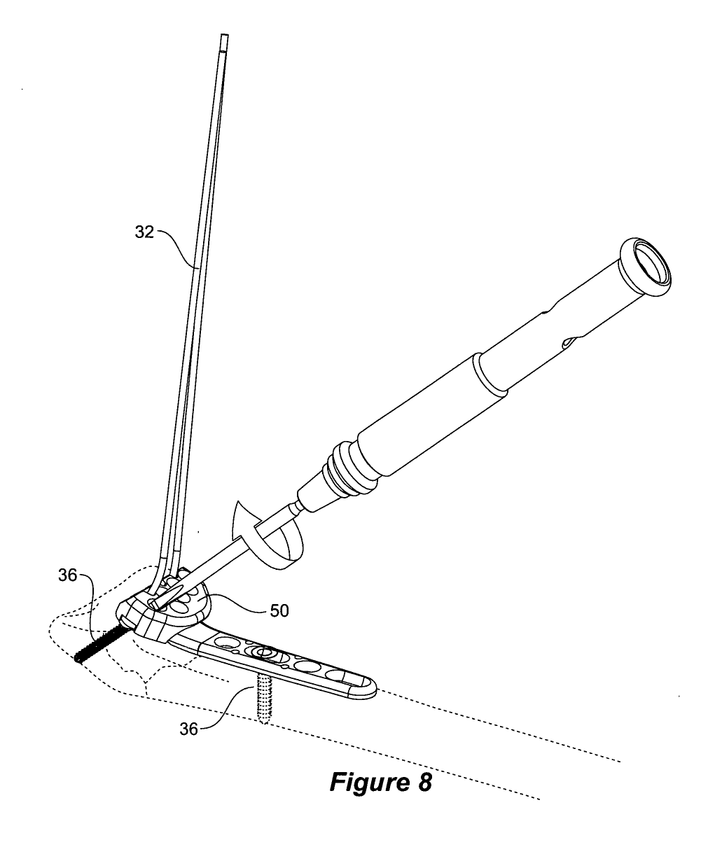

Alternatively, once the holes are drilled in the predetermined position in the bone 30 (ie by drill 35 inserted within the drill sleeve 34 guided by tool jig aperture 54), the method may involve inserting the fixing element 36 through tool jig aperture 54 into aligned fixing element aperture 20 and into the hole that has been drilled to fix the bone plate to the bone 30, as illustrated in FIG. 8. The tool jig 50 may then be removed from the bone plate 10 by inserting a lever into the recessed region 58 and applying a sufficient separating force.

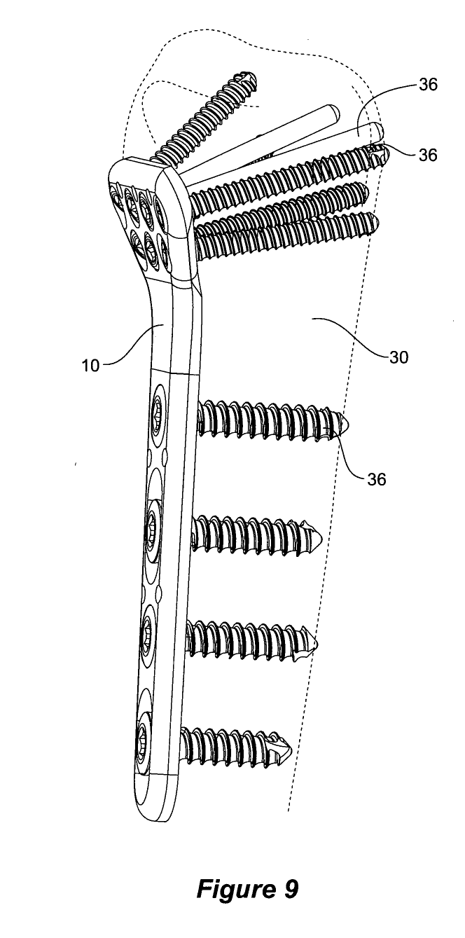

Referring to FIG. 9, there is shown a bone plate 10 fixed to bone 30 by fixing elements 36, following the removal of the tool jig 50. The fixing elements 36 that have passed through fixing element aperture 20 are inserted at predetermined positions and angles that, together with the bone plate 10, optimally enhance healing of a fractured or broken bone.

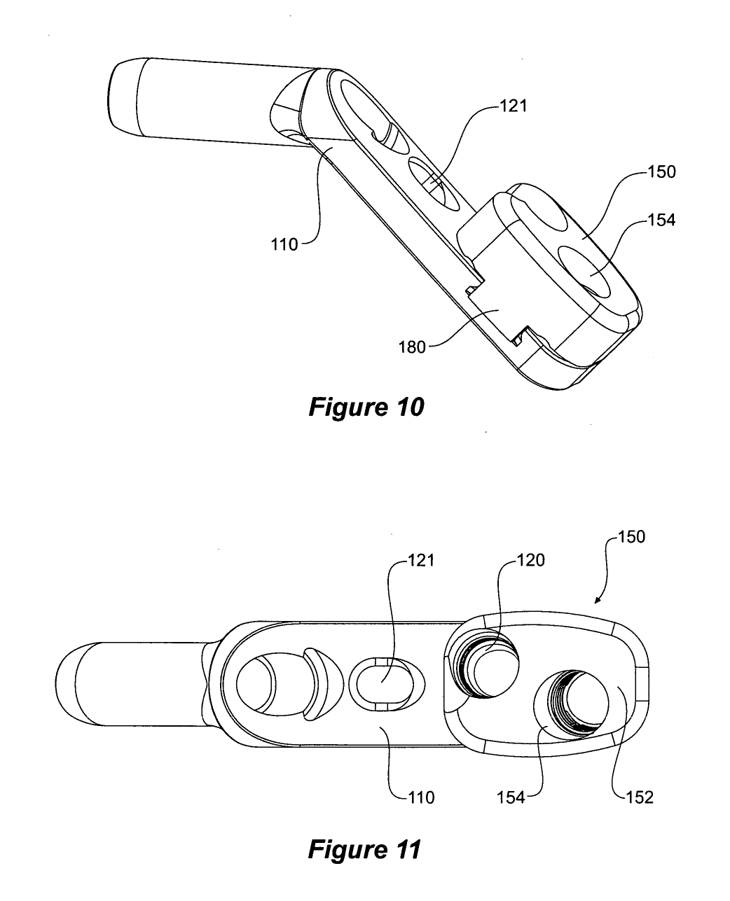

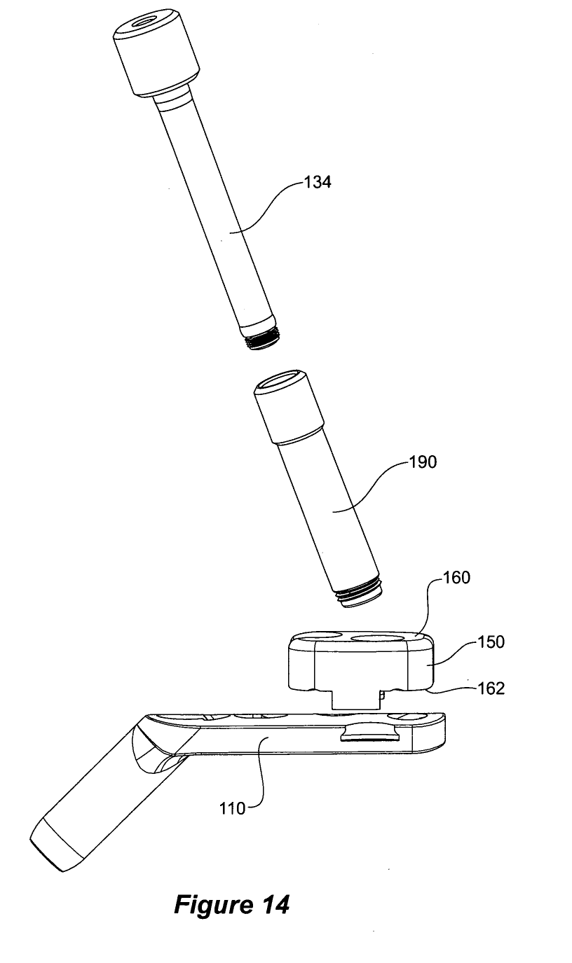

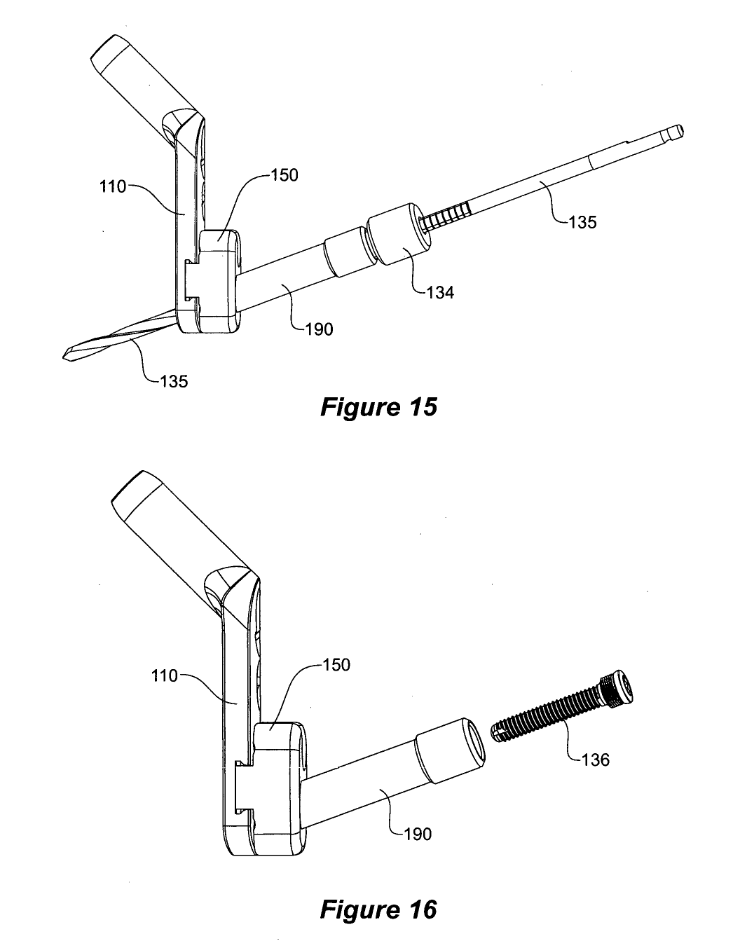

Another embodiment is illustrated in FIGS. 10 to 17, which show a medical implant assembly 105 comprising a tool jig 150 which is releasably secured to a bone plate 110. The bone plate 110 encompasses a plurality of fixing element apertures 120, 121, through which holes can be drilled into underlying bone 130 to enable fixing of the bone plate 110 to bone 130. However, it is desirable to position of fixing elements 136, together with the bone plate 110, at predetermined positions and angles to optimally enhance healing of a fractured or broken bone. The fixing elements 136 may engage bone fragments, fractured bone or non-fractured bone. The fixing element apertures 120,121 can be elongate or circular and sized to fit a range of fixing elements such as but not limited to screws, nails, pins and Kirschner wires. The fixing element apertures 120,121 may be threaded, partially threaded or plain; and they may be shaped or tapered such that fixing elements can be seated flush within the aperture. In the illustrated embodiment, the fixing element apertures 120 in use align with tool jig apertures 154, are circular and adapted to fit a screw. The fixing element apertures 120 receive locking screws as fixing elements, and the fixing element aperture 121 receives a non-locking screw as a fixing element, although other embodiments are possible. The medical assembly 105 may be provisionally attached to the bone with a non-locking screw through fixing element aperture 121 prior to drilling holes through fixing element apertures 120.

The tool jig 150 comprises a body 152 encompassing a plurality of tool jig apertures 154, and a securing means comprising finger members 180. When the tool jig 150 is releasably secured to the bone plate 110, the tool jig apertures 154 precisely align with the predetermined positions and angles of fixing element apertures 120. The illustrated embodiment shows two tool jig apertures 154, which can be angled or normal to the bone plate 110 depending upon the angle of the corresponding fixing element aperture 120. The tool jig apertures 154 generally pass from a top 160 through to a base 162 of the tool jig, although some or all of the tool jig apertures 154 may pass through different surfaces of the tool jig 150 in some embodiments. In the illustrated embodiment, part of one of the tool jig apertures 154 passes through a side surface of the tool jig 150. The tool jig apertures 154 are substantially cylindrical in shape, with an equal diameter at the top 160 and base 162 of the tool jig 150.

The internal diameter of the tool jig apertures 154 is adapted such that, in use, an outer sleeve 190 has a threaded fit within a tool jig aperture 154. The tool jig aperture 154 is threaded to receive the outer sleeve 190. The tool jig aperture 154 guides the outer sleeve 190 so that the longitudinal axis of the outer sleeve 190 substantially aligns with the axis of the aligned fixing element aperture 120. A drill sleeve 134 can be slidably located within the outer sleeve 190 and the drill sleeve 134 can be attached to the aligned fixing element aperture 120 by threading. The outer sleeve 190 guides placement of the drill sleeve 134 within the fixing element aperture 120 so that the longitudinal axis of the drill sleeve 134 substantially aligns with the axis of the fixing element aperture 120 in the bone plate 110 prior to attaching the drill sleeve 134 to the fixing element aperture 120. The fixing element apertures 120 are threaded to receive drill sleeve 134.

Accordingly, the tool jig apertures 154 guide placement of an outer sleeve 190 within the tool jig aperture 154 wherein the outer sleeve 190 further guides placement of the drill sleeve 134 within the fixing element aperture 120. The drill sleeve 134 is firmly held within the fixing element aperture 120 at an angle to enable a hole to be drilled in the correct predetermined position and angle by a drill 135 into the underlying bone 130 in a within the fixing element apertures 120. This assembly with an extra guiding sleeve is particularly useful in situations where it is highly desirable to minimise the possibility of the fixing element cross-threading in the drilled hole, for example, where the fixing elements are relatively long.

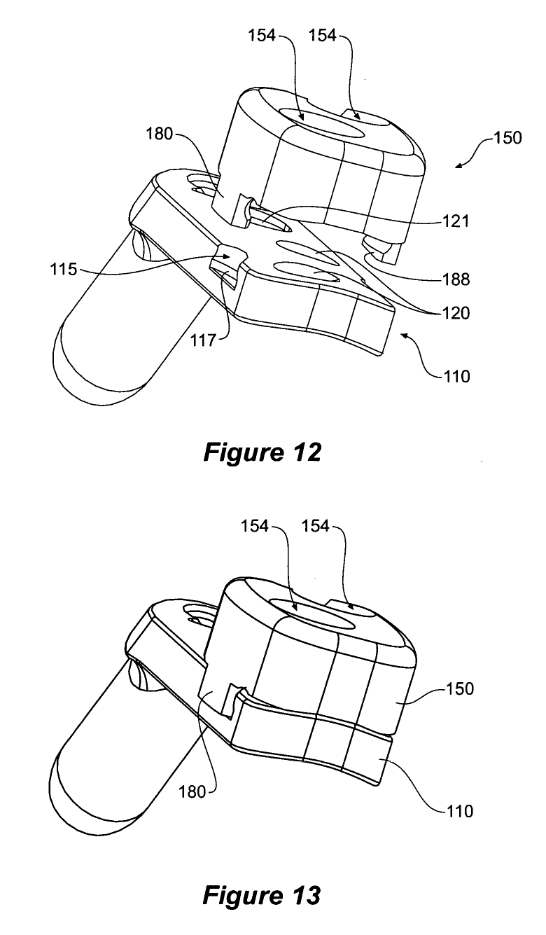

The securing means is adapted to releasably secure the tool jig 150 to the bone plate 110 by clipping, and once the holes are drilled in the predetermined position in the bone 130 (ie by drill 135 inserted within the drill sleeve 134 guided by outer sleeve 190 guided by tool jig aperture 154), the tool jig 150 can be released from the bone plate 110. The securing means includes a plurality of finger members 180 that extend from the body 152 of the tool jig 150 and resiliently engage with the bone plate 10. The engaging surface 115 on the perimeter of bone plate 110 and finger members 180 are contoured to engage with one another to releasably secure the tool jig 150 to the bone plate 110 in a manner that substantially prevents movement of the tool jig 150 relative to the bone plate. Specifically, the interior aspects 182 of the finger members 80 are concavely shaped to conform and engage with the engaging surface 115 of the bone plate 110 such that in use the finger members 180 secure the tool jig 150 to the bone plate 110 by clipping. The finger members 180 are positioned on the perimeter of the body 152 of the tool jig 150. In particular, there are two finger members 180, each located on opposing sides 168, 169 of the tool jig 150. The finger members 180 extend from the tool jig base 162 away from the tool jig body 152, and are integrally formed with the body 152. A securing region is located at the base 186 of each finger member 180, and a securing tooth member 188 projects from the securing region to, in use, engage with the engaging surface 115 of the bone plate 110. As shown in FIG. 12, the engaging surface 115 is a recess on the opposing sides 111, 113 on the perimeter of the bone plate 110. Engaging surface 115 provides a lead-in to an indented securing surface, slot 117, at the lower edge of engaging surface 115, and which is shaped for engagement with securing tooth member 188. Securing tooth members 188 may comprise a flange means that in use engages with the indented securing surface slot 117 of the bone plate 110. In use, the securing means 180 resiliently deforms to deflect outwardly to allow engagement of the tooth member 188 into the indented securing surface slot 117 to thereby secure the tool jig 150 onto the bone plate 110.

The application of some force is required to remove the tool jig 150 from bone plate 110, minimising the possibility of accidental dislodgment during surgery. Once the holes are drilled in the predetermined position in the bone 130 (ie by drill 135 inserted within the drill sleeve 134 guided by outer sleeve 190 guided by tool jig aperture 154), the tool jig 150 may be removed by using the outer sleeve 190, which is threaded into tool jig aperture 154, as a tool that enables separation of the tool jig 150 from the bone plate 110. Accordingly, tool jig aperture 154 also functions as a recessed region adapted to allow engagement with a tool that enables separation of the tool jig 150 from the bone plate 110. A sufficient separating force to separate the tool jig 150 from the bone 130 can be a levering action or a twisting action applied to the outer sleeve 190 to release the securing means by clipping.

FIGS. 10 to 17 illustrate a method of method of fixing the bone plate 110 to a bone 130. The medical implant 105 may be assembled by clipping the tool jig 150 onto the bone plate 110 such that the tooth members 188 of the finger members 180 engage the engaging surface 115 and indented securing surface 117 of the bone plate 110. The medical implant assembly may be sterilised prior to implant surgery. During surgery, the medical implant assembly 105 is arranged against the bone 30. The bone plate 10 may be provisionally engaged with the underlying bone by inserting fixing elements though holes drilled through fixing element aperture 121.

An outer sleeve 190 is threaded into a tool jig aperture 154 wherein the tool jig aperture 154 guides the outer sleeve 190 so that the longitudinal axis of the outer sleeve 190 substantially aligns with the axis of an aligned fixing element aperture 120. An inner drill sleeve 134 is slidably located within the outer sleeve 190, and the drill sleeve 134 is attached to the aligned fixing element aperture 120 by threading. A drill 135 is located to be within the inner drill sleeve 134 and hole is drilled in a predetermined position through fixing element aperture 120 in the bone 130. The inner drill sleeve 134 is removed, and a fixing element 136 is inserted through the outer sleeve 190 and through the tool jig aperture 154, to be received into the aligned fixing element aperture 120 and the hole that has been drilled to fix the bone plate 110 to the bone 130. Finally, the tool jig 150 is removed from the bone plate 110 using the outer sleeve 190 as a tool.

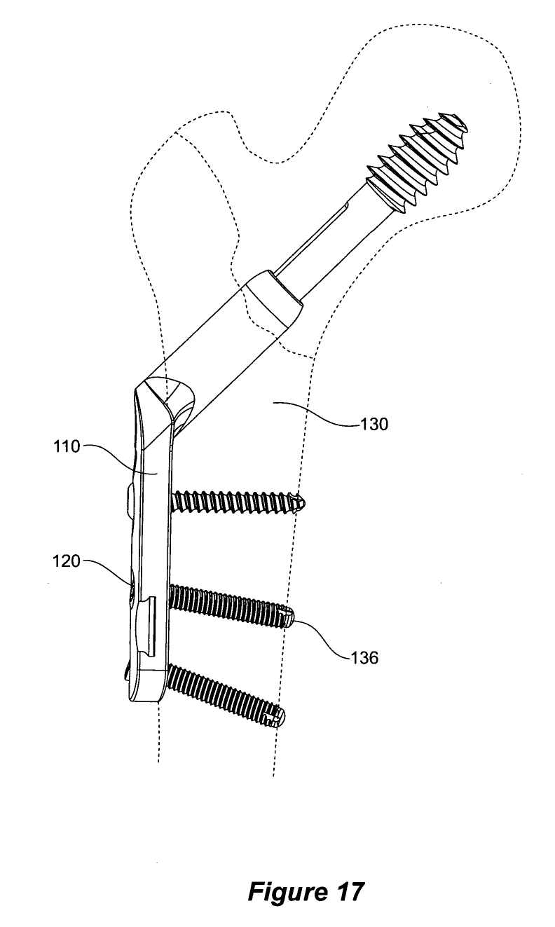

Referring to FIG. 17, there is shown a bone plate 110 fixed to bone 130 by fixing elements 136, following the removal of the tool jig 150. The fixing elements 136 that have passed through fixing element aperture 120 are inserted at predetermined positions and angles that, together with the bone plate 110, optimally enhance healing of a fractured or broken bone.

In the illustrated embodiments, tool jig apertures that are adapted such that, in use, each tool jig aperture is aligned with a predetermined fixing element aperture of a predetermined bone plate. Tool jig 50 as illustrated in FIGS. 3 to 8 is adapted to be used with a fixed angle distal radius bone plate, for example, an Austofix VRP Volar Radius Plate. Tool jig 150 is illustrated adapted to be used with a hip bone plate. Preferably, the hip bone plate is a sliding hip bone plate, for example, an Austofix Tectona Hip Plate.

The tool jig may be fashioned from a polymer or medical grade plastic although the tool jig is not limited to such materials. The preferred material for the fabrication is a thermoplastic (such as but not limited to polyphenylsulfone) as it is gamma stable and medically approved for instrumentation. A thermoplastic tool jig is advantageous as it is resilient with good shape memory enabling a tool jig to be easily clipped on and off a bone plate without damaging the finger members of the tool jig, the body of the tool jig or the bone plate. Metallic tool jigs on the other hand are less desirable as they may damage underlying metallic bone plates, in particular the oxide coating of implanted metallic implants, anodising the implant and thus posing a risk of mechanical fatigue of the bone plate.

The tool jig may be disposable and can be disposed of once it the bone plate has been secured to bone. The tool jig, medical implant assembly or kit of the present invention may be sterile and contained within sterile packaging ready for use in surgery. The medical implant assembly and/or kit of the invention may also be sterile and contained within sterile packaging ready for use in surgery.

In an embodiment, the drill sleeve and the bone plate can be arranged such that the drill sleeve extends a predetermined distance from the bone plate though the tool jig aperture once the drill sleeve has been completely screwed in and tightened. This substantially results in a predetermined drilling depth in which fixing elements of a predetermined length can be used to fix the bone plate to underlying bone.

In another embodiment of a medical implant assembly (not illustrated), the tool jig apertures are non-threaded and the fixing element apertures are non-threaded. In said embodiment, a drill sleeve can be pushed though the tool jig apertures and into the fixing element apertures. Movement of the drill sleeve through the fixing element aperture can be limited by a shoulder within fixing element aperture. This substantially results in a constant depth for fixing elements used to secure the bone plate to underlying bone.

It will be understood that the term “comprise” and any of its derivatives (eg. comprises, comprising) as used in this specification is to be taken to be inclusive of features to which it refers, and is not meant to exclude the presence of any additional features unless otherwise stated or implied.

Although illustrative embodiments of the present invention have been described in the foregoing detailed description, it will be understood that the invention is not limited to the embodiments disclosed, but is capable of numerous rearrangements, modifications and substitutions without departing from the scope of the invention as set forth and defined by the following claims.

1. A tool jig for use with a bone plate having one or more fixing element apertures, wherein the tool jig guides placement of a drill sleeve to enable the drill sleeve to be attached to a fixing element aperture, the tool jig comprising
a body,
a securing means on the body adapted to releasably secure the tool jig to the bone plate such that the tool jig can be firstly secured in position to the bone plate and secondly removed, and
one or more tool jig apertures defined in the body wherein an at least one or all tool jig apertures are adapted to be substantially aligned with an at least one or all fixing element apertures;
wherein, in use, the tool jig aperture guides the drill sleeve so that the longitudinal axis of the drill sleeve substantially aligns with the axis of the fixing element aperture prior to attaching the drill sleeve to the fixing element aperture.

2. The tool jig according to claim 1 wherein the drill sleeve is a cylindrical drill sleeve for guiding a drill to drill a hole in bone.

3. The tool jig according to claim 1 or 2 wherein the securing means extends from the body and is adapted to, in use, resiliently engage with the bone plate by clipping to releasably secure the tool jig to the bone plate.

4. The tool jig according to any one of claims 1 to 3 wherein the securing means extends from the body to a securing region having at least one securing tooth member that projects from the securing region to, in use, engage with an engaging surface of the bone plate to releasably secure the tool jig to the bone plate.

5. The tool jig according to claim 4 wherein at least two opposed securing tooth members each comprise a flange means, in use, engage with an indented securing surface of the bone plate to releasably secure the tool jig to the bone plate.

6. The tool jig according to claim 4 or 5 wherein the securing means comprises a plurality of finger members.

7. The tool jig according to any one of claims 1 to 6 wherein the tool jig further comprises a recessed region that, in use, is adapted to allow engagement with a tool that enables separation of the tool jig from the bone plate.

8. The tool jig according to claim 7 wherein the recessed region is located at the base of the tool jig.

9. The tool jig according to claim 7 wherein the recessed region is at least one of the tool jig apertures.

10. The tool jig according to any one of claims 1 to 9 wherein at least one of the tool jig apertures further guides placement of an outer sleeve within the tool jig aperture, wherein the outer sleeve further guides placement of the drill sleeve within the fixing element aperture.

11. The tool jig of any one of claims 1 to 10 wherein at least one or all of the tool jig apertures have an internal diameter that is adapted to have a sliding fit with the drill sleeve or an outer sleeve for guiding a drill sleeve.

12. The tool jig according to any one of claims 1 to 10 wherein at least one or all of the tool jig apertures are at least partially threaded to engage the drill sleeve or an outer sleeve for guiding a drill sleeve.

13. The tool jig according to any one of claims 1 to 12 wherein at least one or all of the tool jig apertures have an internal diameter that is adapted to be larger than the external diameter of a fixing element such that, in use, the fixing element can pass through the tool jig aperture to be seated within the body plate aperture.

14. The tool jig according to any one of claims 1 to 13 wherein the tool jig apertures are adapted such that, in use, each tool jig aperture is aligned with a predetermined fixing element aperture of a predetermined bone plate.

15. The tool jig according to any one of claims 1 to 14 wherein the tool jig is adapted to be used with a bone plate selected from a fixed angle distal radius bone plate or a hip bone plate.

16. The tool jig according to any one of claims 1 to 15 wherein the tool jig is formed of a polymer.

17. The tool jig according to claim 16 wherein the tool jig is formed of polyphenylsulfone.

18. A medical implant assembly comprising:
a tool jig according to any one of claims 1 to 17, and
a bone plate having one or more fixing element apertures adapted to receive a drill sleeve;
wherein the tool jig aperture acts to guide the drill sleeve so that the longitudinal axis of the drill sleeve substantially aligns with the axis of the fixing element aperture prior to attaching the drill sleeve to the fixing element aperture.

19. The medical assembly according to claim 18 wherein the bone plate is selected from a fixed angle distal radius bone plate or a hip bone plate.

20. A kit comprising the medical implant assembly according to claim 18 or 19 and at least one drill sleeve.

21. A method of fixing a bone plate to a bone comprising the following steps:
(a) arranging the medical implant assembly of claim 18 or 19 against a bone;
(b) inserting a drill sleeve into a tool jig aperture and attaching the drill sleeve to the fixing element aperture wherein the tool jig aperture guides the drill sleeve so that the longitudinal axis of the drill guide substantially aligns with the axis of an aligned fixing element aperture;
(c) drilling a hole in a predetermined position in the bone by locating the drill within the drill sleeve;
(d) removing the tool jig from the bone plate; and
(e) inserting a fixing element into the fixing element aperture and the hole that has been drilled to fix the bone plate to the bone.

22. A method for fixing a bone plate to a bone comprising the following steps:
(a) arranging the medical implant assembly of claim 18 or 19 against a bone;
(b) inserting a drill sleeve into a tool jig aperture and attaching the drill sleeve to the fixing element aperture wherein the tool jig aperture guides the drill sleeve so that the longitudinal axis of the drill guide substantially aligns with the axis of an aligned fixing element aperture;
(c) drilling a hole in a predetermined position in the bone by locating the drill within the inner drill sleeve;
(d) removing the drill sleeve;
(e) inserting a fixing element into the fixing element aperture and the hole that has been drilled to fix the bone plate to the bone; and
(f) removing the tool jig from the bone plate.

23. A method for fixing a bone plate to a bone comprising the following steps:
(a) arranging the medical implant assembly of claim 18 or 19 against a bone;
(b) threading an outer sleeve into a tool jig aperture wherein the tool jig aperture guides the outer sleeve so that the longitudinal axis of the outer sleeve substantially aligns with the axis of an aligned fixing element aperture;
(c) slidably locating an inner drill sleeve within the outer sleeve and attaching the drill sleeve to the aligned fixing element aperture;
(d) drilling a hole in a predetermined position in the bone by locating the drill within the inner drill sleeve;
(e) removing the inner drill sleeve;
(f) inserting a fixing element through the outer sleeve and into the aligned fixing element aperture and the hole that has been drilled to fix the bone plate to the bone; and
(g) removing the tool jig from the bone plate using the outer sleeve as a tool.

24. A tool jig substantially as herein described with reference to any one of the embodiments of the invention illustrated in the accompanying drawings.
